(19) United States
(12) Patent Application Publication
(10) Pub. No.: US 2026/0125714 A1
(43) Pub. Date: May 7, 2026

(54) METHANE CONVERSION SYSTEM AND METHODS THEREOF

(71) Applicant: ExxonMobil Technology and Engineering Company, Spring, TX (US)

(72) Inventors: Lin ZHAO, Spring, TX (US); Sam AMINFARD, Houston, TX (US); Jordan C. ANGLE, Tomball, TX (US)

(21) Appl. No.: 19/117,541

(22) PCT Filed: Sep. 29, 2023
(86) PCT No.: PCT/US2023/075523

§ 371 Date: Apr. 1, 2025

Related U.S. Application Data
(60) Provisional application No. 63/379,333, filed on Oct. 13, 2022.

(51) Int. Cl. C12P 1/04; C12M 1/107

(52) U.S. Cl. CPC C12P 1/04; C12M 21/04

(57) ABSTRACT

A method for methane oxidation by methanotrophic bacteria to reduce methane emissions may include: conveying a gas comprising methane to a methane conversion system that comprises a bed of oxic soil containing methanotrophic bacteria; contacting the methanotrophic bacteria and the gas; and allowing the methanotrophic bacteria to oxidize at least a portion of the methane to carbon dioxide. The gas stream may have about about 100 ppm to about 10,000 ppm methane present.

### CROSS-REFERENCE TO RELATED APPLICATION

This application claims the benefit of U.S. Provisional Application Ser. No. 63/379,333, entitled “METHANE CONVERSION SYSTEM AND METHODS THEREOF,” filed Oct. 13, 2022, the disclosure of which is hereby incorporated by reference in its entirety.

### FIELD OF INVENTION

The present disclosure relates to methane oxidation, and more particularly, methane oxidation by methanotrophic bacteria to reduce methane emissions.

### BACKGROUND

Methane is the second most abundant anthropogenic greenhouse gas after carbon dioxide, accounting for approximately 20% of global emissions. Although less abundant, methane is significantly more potent than carbon dioxide in terms of global warming potential because methane is estimated to be more than 25 times as effective as carbon dioxide at trapping heat in the atmosphere on a 100-year basis. Over the last two centuries, methane concentrations in the atmosphere have more than doubled, largely due to human-related activities, such as landfill use, oil and gas production and processing, agricultural activities, coal mining, stationary and mobile combustion, wastewater treatment, and other industrial processes. Because methane is a powerful greenhouse gas, a reduction in methane emissions would have a rapid and significant effect on the atmospheric warming potential.

Several methods to reduce methane emissions have been suggested, the simplest method being a reduction or elimination of the processes that use or produce methane. However, reducing or eliminating processes producing or utilizing methane is often infeasible and costly. In situations where the use or production of methane is necessary, other methods of methane mitigation, such as the conversion of methane into less environmentally-harmful species, are utilized. There are various techniques to convert methane including (i) reforming to produce syngas, including steam reforming, dry reforming, and partial oxidation; (ii) oxidative coupling; and (iii) conversion to oxygenates such as methanol. However, the metal catalysts commonly used in these techniques can be costly to produce and use, making these processes economically unattractive and creating a gap in the demand for methane emission control.

### SUMMARY OF INVENTION

A nonlimiting example method of the present disclosure may comprise: conveying a gas comprising methane to a methane conversion system that comprises a bed of oxic soil containing methanotrophic bacteria; wherein methane is present in the gas stream at about 100 ppm to about 10,000 ppm; contacting the methanotrophic bacteria and the gas; and allowing the methanotrophic bacteria to oxidize at least a portion of the methane to carbon dioxide.

A nonlimiting example methane conversion system of the present disclosure may comprise: a source that produces a gas stream having a methane concentration from about 100 ppm to about 10,000 ppm; oxic soil containing methanotrophic bacteria, wherein the methanotrophic bacteria are capable of oxidizing at least a portion of the methane to carbon dioxide; and a gas distribution system configured to receive the gas stream and distribute the gas stream through the oxic soil.

These and other features and attributes of the disclosed methods and systems of the present disclosure and their advantageous applications and/or uses will be apparent from the detailed description which follows.

### BRIEF DESCRIPTION OF THE DRAWINGS

To assist those of ordinary skill in the relevant art in making and using the subject matter hereof, reference is made to the appended drawings. The following figures are included to illustrate certain aspects of the disclosure, and should not be viewed as exclusive configurations. The subject matter disclosed is capable of considerable modifications, alterations, combinations, and equivalents in form and function, as will occur to those skilled in the art and having the benefit of this disclosure.

FIG. 1 illustrates an example of a methane conversion system of the present disclosure.

FIG. 2 illustrates another example of a methane conversion system of the present disclosure.

### DETAILED DESCRIPTION

The present disclosure relates to methane oxidation, and more particularly, methane oxidation by methanotrophic bacteria to reduce methane emissions. Methanotrophic bacteria—microorganisms that metabolize methane gas—are ubiquitous in nature, where the bacteria utilize oxygen and water to oxidize methane. This process has been shown to play an essential role in landfill methane oxidation, oxidizing up to 100% of the methane entering shallow, cap-layer soils.

Methanotrophs can live under aerobic or anaerobic conditions. Aerobic methanotrophs combine oxygen and methane to form carbon dioxide and formaldehyde, the latter being incorporated into organic compounds via the serine pathway or the ribulose monophosphate pathway. Conversely, anaerobic methanotrophs use electron acceptors other than oxygen for methane oxidation.

In one form of the present disclosure, naturally-occurring oxic soil comprising methanotrophic bacteria may be utilized to reduce methane emissions from natural gas-fueled engines or other methane sources. In one example, layers of oxic soil beds comprising methanotrophic bacteria may be stacked together with gas diffusers beneath each oxic soil bed layer to facilitate the diffusion of a gas stream comprising methane through the oxic soil. The stacked layers of oxic soil beds may be enclosed in a housing unit. In another example, vessel (above ground and/or beneath ground) may be utilized to store a gas stream comprising methane before distributing the gas through the ground comprising oxic soil containing methanotrophic bacteria. Such a vessel may allow for an inflow thereto to be different from an outflow therefrom, which allows for the sources to produce the gas stream comprising methane at a first rate that may be different than a rate at which the gas stream comprising methane is distributed to the oxic soil containing methanotrophic bacteria.

### Definitions

As used in the present disclosure and claims, the singular forms “a,” “an,” and “the” include plural forms unless the context clearly dictates otherwise.

The term “and/or” as used in a phrase such as “A and/or B” herein is intended to include “A and B,” “A or B,” “A,” and “B.”

The term “lean-burn reciprocating engine” refers to an engine having an air-to-fuel equivalence ratio greater than one.

The term “methanotroph” and grammatical derivatives thereof refer to prokaryotes that metabolize and/or oxidize methane as a source of energy.

As used herein, the terms “optional” or “optionally” mean that the subsequently described event or circumstance can or cannot occur and that the description includes instances where said event or circumstance occurs and instances where it does not.

The term “oxic soil” refers to a soil that has a detectable amount of oxygen (e.g., at least 300 ppm oxygen) in the soil atmosphere when measured by a soil oxygen sensor.

As used herein, the terms “soil bed” and “bed” refer to a layer of soil that may be optionally contained within a containment structure.

### Methane Conversion Systems and Methods

The present disclosure provides methods and systems for the conversion of methane by methanotrophic bacteria. For example, methane conversion may be achieved by a method comprising conveying a gas (also referred to herein as a gas stream) comprising methane to a methane conversion system, said methane conversion system comprising an oxic soil containing methanotrophic bacteria, and causing the methanotrophic bacteria and the gas to come into contact. The methanotrophic bacteria within the oxic soil may oxidize at least a portion of the methane to carbon dioxide, thereby reducing the global warming potential of the gas.

Methane may be present in the gas (or gas stream) at a concentration of about 100 ppm to about 10,000 ppm (or about 100 ppm to about 1000 ppm, or about 500 ppm to about 2000 ppm, or about 1000 ppm to about 5000 ppm, or about 2500 ppm to about 7500 ppm, or about 5000 ppm to about 10,000 ppm).

The source of the gas stream may, for example, be a lean-burn reciprocating engine. Said lean-burn reciprocating engine may be capable of about 500 horsepower (hp) to about 5000 hp (or about 500 hp to about 1500 hp, or about 600 hp to about 1600 hp, or about 700 hp to about 1700 hp, or about 800 hp to about 1800 hp, or about 900 hp to about 1900 hp, or about 1000 hp to about 2000 hp, or about 1000 hp to about 5000 hp). The lean-burn reciprocating engine may be from an onshore gas compression station. Other sources of the gas stream include, but are not limited to, rich-burn engines, crankcase venting streams, gas processing plant vents, coal mine vents, the like, and any combination thereof.

The temperature of the gas stream exiting the source may be higher than the temperature at which the methane conversion takes place. The temperature of the gas stream exiting the source may, for example, be about 100° C. to about 1000° C. (or about 100° C. to about 500° C., or about 250° C. to about 750° C., or about 300° C. to about 800° C., or about 500° C. to about 1000° C.). Before contact with the methanotrophic bacteria, the gas stream may be cooled to ambient temperature (e.g., about 25° C.) or to about 20° C. to about 45° C. (or about 20° C. to about 30° C., or about 25° C. to about 35° C., or about 30° C. to about 40° C., or about 35° C. to about 45° C.). Cooling of the gas stream may be achieved using a cooling system, a waste heat recovery system, the like, and any combination thereof.

Methanotrophic bacteria suitable for use in the methane conversion systems and methods of the present disclosure may be aerobic methanotrophic bacteria. Examples of aerobic methanotrophic bacteria may include, but are not limited to, Methylomonas aurantiaca, Methylomonas fodinarum, Methylomonas methanica, Methylobacter albus, Methylobacter marinus, Methylobacter whittenburyi, Methylococcus capsulatus, Methylococcus thermophiles, Methylomicrobium agile, Methylomicrobium album, Methylomicrobium pelagicum, the like, and any combination thereof. The methanotrophic bacteria may be native microbes of oxic soil.

The methanotrophic bacteria may, for example, have a methane conversion capacity of about 0.01% to about 10% (or about 0.01% to about 5%, or about 1% to about 6%, or about 2% to about 7%, or about 3% to about 8%, or about 4% to about 9%, or about 5% to about 10%) based on the ratio of the volume of methane to the volume of the oxic soil in which the methanotrophic bacteria are located.

Methane oxidation by the methanotrophic bacteria may, for example, occur at about 20° C. to about 45° C. (or about 20° C. to about 30° C., or about 25° C. to about 35° C., or about 30° C. to about 40° C., or about 35° C. to about 45° C.). The optimal oxidation temperature may be 37° C.

The methanotrophic bacteria suitable for use in the methane conversion systems and methods of the present disclosure may co-oxidize volatile organic compounds other than methane. Examples of volatile organic compounds other than methane include, but are not limited to, tetrachloromethane, trichloromethane, dichloromethane, trichloroethylene, vinyl chloride, benzene, toluene, methanol, the like, and any combination thereof.

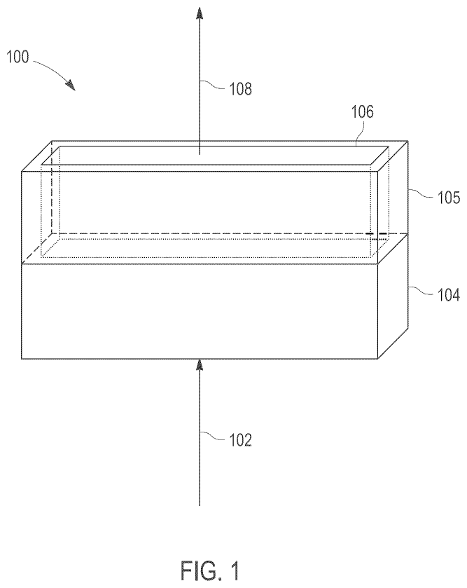

FIG. 1 illustrates a nonlimiting example of a methane conversion system 100 of the present disclosure. The methane conversion system 100 may include a feed line 102 used in conveying a gas stream comprising methane to a gas distribution system 104. The gas distribution system 104 in turn distributes the gas stream through a bed 106 of oxic soil containing methanotrophic bacteria. The methanotrophic bacteria in the oxic soil bed 106 convert at least a portion of the methane in the gas stream, releasing an effluent stream 108 lower in methane concentration than the entering gas stream in the feed line 102.

The gas distribution system 104 may be used to distribute the gas stream in the feed line 102 through the bed 106 containing the methanotrophic bacteria and may, for example, comprise a gas diffuser, a nozzle, a valve, a manifold, the like, or any combination thereof. The feed line 102 may additionally comprise therealong one or more compressors (e.g., upstream of the gas distribution system 104) to convey the gas stream to the gas distribution system 104.

The bed 106 may include a containment structure 105 that physically contains the oxic soil. Examples of the containment structure 105 may include, but are not limited to, a tray, a box, a trough, a crate, a planter, the like, and any combination thereof. The containment structure 105 may be at least partially formed by materials that may include, but are not limited to, wood, metal, plastic, paper, the like, and any combination thereof.

One or more openings (not illustrated) may be present in the containment structure 105 to facilitate the movement of gas in and out of the bed 106. For example, such openings may facilitate the diffusion of carbon dioxide and oxygen between the bed 106 and the environment around the bed.

The bed 106 may, for example, have a bed depth ranging from about 20 cm deep to about 50 cm deep (or about 20 cm to about 30 cm, or about 25 cm to about 35 cm, or about 30 cm to about 40 cm, or about 35 cm to about 45 cm, or about 40 cm to about 50 cm).

The bed 106 may, for example, have a cross-sectional area perpendicular to the flow direction of the gas stream in the feed line 102 of about 100 cm² to about 50 m² (or about 100 cm² to about 2500 cm², or about 1000 cm² to about 10,000 cm², or about 0.1 m² to about 5 m², or about 1 m² to about 25 m², or about 1 m² to about 50 m²).

To enhance the efficiency of the methane conversion, system 100 may, for example, comprise a plurality of gas distribution systems 104 and oxic soil beds 106. When a plurality of gas distribution systems 104 and beds 106 are used, the gas distribution systems 104 and beds 106 may be stacked vertically on top of one another (e.g., from bottom to top: (i) first gas distribution system, first bed, second gas distribution system, second bed, third gas distribution system, and third bed, or (ii) first gas distribution system, first bed, second bed, second gas distribution system, third bed, fourth bed, or (iii) other stacked configurations). System 100 may comprise a plurality of stacks of gas distribution systems 104 and beds 106 in parallel.

The effluent stream 108 may comprise carbon dioxide converted from methane by the methanotrophic bacteria, optionally other VOC conversion products from the methanotrophic bacteria, and unconverted species of the gas stream not converted by the methanotrophic bacteria to other species. The effluent stream 108 may, for example, be vented to the atmosphere or transported to other units for further processing before being vented to the atmosphere.

The bed 106 may be open to the environment in at least one location (e.g., at the top) to allow for the effluent stream 108 to enter the surrounding environment. Alternatively, bed 106 may be enclosed and have a line for conveying the effluent stream 108 to another location before venting to the atmosphere and/or other processing.

The carbon dioxide produced by the methanotrophic bacteria may also be used to support the growth of other organic matter.

The methanotrophic bacteria may, for example, produce biomass in addition to carbon dioxide.

FIG. 1 is a general illustration and other components may be included in the system to ensure the proper and safe operation thereof. Additional components may include, but are not limited to, valves, heat exchangers, pressure meters, flow rate meters, sensors (e.g., pressure sensors, temperature sensors, flow rates sensors), pumps, additional lines (e.g., pipes or conduits for flowing fluids), and the like, and combinations thereof.

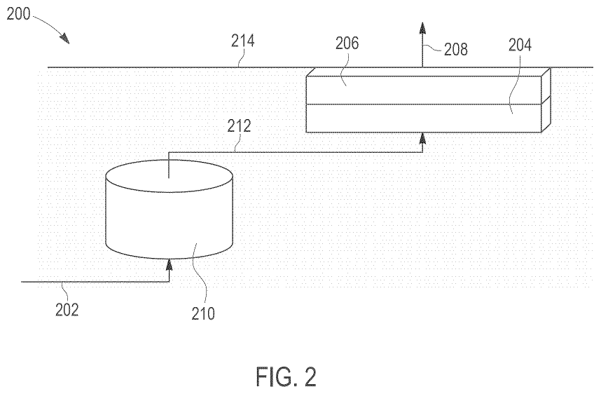

FIG. 2 illustrates another nonlimiting example of a methane conversion system 200 of the present disclosure. A feed line 202 is used in conveying a gas stream comprising methane to a vessel 210 where the gas stream may be passed through and/or stored. The feed line 202 may comprise a valve to prevent the backflow of gas to the source. From the vessel 210, which is downstream of the source and upstream of the gas distribution system 204, the gas stream is directed to a gas distribution system 204 via line 212. The gas distribution system 204 distributes the gas stream through a bed 206 of oxic soil containing methanotrophic bacteria. The effluent stream 208 may have a lower concentration of methane than the entering gas stream of feed line 202 and line 212.

The illustrated gas distribution system 204 is beneath ground level 214, and the bed 206 is at and beneath ground level 214. The gas distribution system may be at ground level, above ground level, below ground level, or a combination thereof. Further, the bed may be at ground level, above ground level, below ground level, or a combination thereof.

The vessel 210 may be at ground level, above ground level, below ground level, or a combination thereof. As illustrated, the vessel 210 (or underground vessel) is below ground level. The top of the vessel 210 may, for example, be below ground level at a depth of about 0.5 m to about 12 m (or about 0.5 m to about 10 m, or about 1 m to about 11 m, or about 2 m to about 12 m).

The vessel 210 may, for example, be about 1 m³ to about 150 m³ in volume (or about 1 m³ to about 50 m³, or about 25 m³ to about 75 m³, or about 50 m³ to 100 m³, or about 75 m³ to 125 m³, or about 100 m³ to 150 m³).

The gas distribution system 204 may comprise several outlets to distribute the gas stream through the bed 206. For example, the gas distribution system 204 may include, but is not limited to, a gas diffuser, a nozzle, a valve, a manifold, the like, or any combination thereof. The feed line 202 and/or the line 212 may additionally comprise therealong one or more compressors (e.g., upstream of the gas distribution system 204, upstream of the vessel 210, downstream of the vessel 210) to convey the gas stream to the vessel 210 and/or to the gas distribution system 204.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the incarnations of the present inventions. At the very least, and not as an attempt to limit the application of the doctrine of equivalents to the scope of the claim, each numerical parameter should at least be construed in light of the number of reported significant digits and by applying ordinary rounding techniques.

One or more illustrative incarnations incorporating one or more invention elements are presented herein. Not all features of a physical implementation are described or shown in this application for the sake of clarity. It is understood that in the development of a physical embodiment incorporating one or more elements of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

### Additional Embodiments

Embodiment 1. A method comprising: conveying a gas comprising methane to a methane conversion system that comprises a bed of oxic soil containing methanotrophic bacteria; wherein methane is present in the gas stream at about 100 ppm to about 10,000 ppm; contacting the methanotrophic bacteria and the gas; and allowing the methanotrophic bacteria to oxidize at least a portion of the methane to carbon dioxide.

Embodiment 2. The method of Embodiment 1, wherein the carbon dioxide is vented to the atmosphere.

Embodiment 3. The method of any one of Embodiments 1-2, wherein the gas stream originates from a lean-burn reciprocating engine.

Embodiment 4. The method of Embodiment 3, wherein the lean-burn reciprocating engine is capable of about 500 hp to about 5000 hp.

Embodiment 5. The method of any one of Embodiments 1-4, wherein the methanotrophic bacteria comprise Methylomonas aurantiaca, Methylomonas fodinarum, Methylomonas methanica, Methylobacter albus, Methylobacter marinus, Methylobacter whittenburyi, Methylococcus capsulatus, Methylococcus thermophiles, Methylomicrobium agile, Methylomicrobium album, Methylomicrobium pelagicum, or any combination thereof.

Embodiment 6. The method of any one of Embodiments 1-5, wherein the methanotrophic bacteria are native microbes of the oxic soil.

Embodiment 7. The method of any one of Embodiments 1-6, wherein the methane conversion system further comprises a containment structure for the bed.

Embodiment 8. The method of any one of Embodiments 1-7, wherein the bed has a bed depth of about 20 cm deep to about 50 cm deep.

Embodiment 9. The method of any one of Embodiments 1-8, wherein the methane conversion system comprises a gas diffuser configured to distribute the gas stream through the oxic soil.

Embodiment 10. The method of any one of Embodiments 1-9, wherein the methane conversion system comprises an underground vessel in which the gas stream is passed through and/or stored before contact with the methanotrophic bacteria.

Embodiment 11. The method of any one of Embodiments 1-10, wherein the methane conversion system further comprises a compressor.

Embodiment 12. The method of any one of Embodiments 1-11, wherein the methanotrophic bacteria have a methane conversion capacity of about 0.01% to about 10% based on the ratio of the volume of methane to the volume of the oxic soil in which the methanotrophic bacteria are located.

Embodiment 13. The method of any one of Embodiments 1-12, wherein the methanotrophic bacteria co-oxidize tetrachloromethane, trichloromethane, dichloromethane, trichloroethylene, vinyl chloride, benzene, toluene, methanol, or any combination thereof.

Embodiment 14. The method of any one of Embodiments 1-13, wherein the methane oxidation occurs at about 20° C. to about 45° C.

Embodiment 15. A methane conversion system comprising: a source that produces a gas stream having a methane concentration from about 100 ppm to about 10,000 ppm; oxic soil containing methanotrophic bacteria, wherein the methanotrophic bacteria are capable of oxidizing at least a portion of the methane to carbon dioxide; and a gas distribution system configured to receive the gas stream and distribute the gas stream through the oxic soil.

Embodiment 16. The methane conversion system of Embodiment 15, wherein the source is from a lean-burn reciprocating engine.

Embodiment 17. The methane conversion system of Embodiment 16, wherein the lean-burn reciprocating engine is capable of about 500 hp to about 5000 hp.

Embodiment 18. The methane conversion system of one or more of Embodiments 15-17, wherein the methanotrophic bacteria comprise Methylomonas aurantiaca, Methylomonas fodinarum, Methylomonas methanica, Methylobacter albus, Methylobacter marinus, Methylobacter whittenburyi, Methylococcus capsulatus, Methylococcus thermophiles, Methylomicrobium agile, Methylomicrobium album, Methylomicrobium pelagicum, or any combination thereof.

Embodiment 19. The methane conversion system of one or more of Embodiments 15-18, wherein the methanotrophic bacteria are native microbes of the oxic soil.

Embodiment 20. The methane conversion system of one or more of Embodiments 15-19, further comprising a containment structure for the bed.

Embodiment 21. The methane conversion system of one or more of Embodiments 15-20, wherein the bed has a bed depth of about 20 cm deep to about 50 cm deep.

Embodiment 22. The methane conversion system of one or more of Embodiments 15-21, wherein the gas distribution system comprises a gas diffuser.

Embodiment 23. The methane conversion system of one or more of Embodiments 15-22, further comprising an underground vessel in which the gas stream is passed through and/or stored, wherein the underground vessel is downstream of the source and upstream of the gas distribution system.

Embodiment 24. The methane conversion system of one or more of Embodiments 15-23, further comprising a compressor upstream of the gas distribution system.

Embodiment 25. The methane conversion system of one or more of Embodiments 15-24, wherein the methanotrophic bacteria have a methane conversion capacity of about 0.01% to about 10% based on the ratio of the volume of methane to the volume of the oxic soil in which the methanotrophic bacteria are located.

Embodiment 26. The methane conversion system of one or more of Embodiments 15-25, wherein the methanotrophic bacteria co-oxidize tetrachloromethane, trichloromethane, dichloromethane, trichloroethylene, vinyl chloride, benzene, toluene, methanol, or any combination thereof.

Embodiment 27. The methane conversion system of one or more of Embodiments 15-26, wherein the methane oxidation occurs at about 20° C. to about 45° C.

The particular examples and configurations disclosed above are illustrative only, as the present invention may be modified and practiced in different but equivalent manners apparent to those skilled in the art and having the benefit of the teachings herein. Furthermore, no limitations are intended to the details of construction or design herein shown, other than as described in the claims below. It is therefore evident that the particular illustrative examples disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

The invention claimed is:

1. A method comprising:
conveying a gas comprising methane to a methane conversion system that comprises an oxic soil containing methanotrophic bacteria, wherein methane is present in the gas stream at about 100 ppm to about 10,000 ppm;
contacting the methanotrophic bacteria and the gas; and
allowing the methanotrophic bacteria to oxidize at least a portion of the methane to carbon dioxide.

2. The method of claim 1, wherein the carbon dioxide is vented to the atmosphere.

3. The method of claim 1, wherein the gas stream originates from a lean-burn reciprocating engine.

4. The method of claim 3, wherein the lean-burn reciprocating engine is capable of about 500 hp to about 5000 hp.

5. The method of claim 1, wherein the methanotrophic bacteria comprise Methylomonas aurantiaca, Methylomonas fodinarum, Methylomonas methanica, Methylobacter albus, Methylobacter marinus, Methylobacter whittenburyi, Methylococcus capsulatus, Methylococcus thermophiles, Methylomicrobium agile, Methylomicrobium album, Methylomicrobium pelagicum, or any combination thereof.

6. The method of claim 1, wherein the methanotrophic bacteria are native microbes of the oxic soil.

7. The method of claim 1, wherein the methane conversion system further comprises a containment structure for the bed.

8. The method of claim 1, wherein the bed has a bed depth of about 20 cm deep to about 50 cm deep.

9. The method of claim 1, wherein the methane conversion system comprises a gas diffuser configured to distribute the gas stream through the oxic soil.

10. The method of claim 1, wherein the methane conversion system further comprises an underground vessel in which the gas stream is passed through and/or stored before contact with the methanotrophic bacteria.

11. The method of claim 1, wherein the methanotrophic bacteria have a methane conversion capacity of about 0.01% to about 10% based on the ratio of the volume of methane to the volume of the oxic soil in which the methanotrophic bacteria are located.

12. The method of claim 1, wherein the methanotrophic bacteria co-oxidize tetrachloromethane, trichloromethane, dichloromethane, trichloroethylene, vinyl chloride, benzene, toluene, methanol, or any combination thereof.

13. The method of claim 1, wherein the methane oxidation occurs at about 20° C. to about 45° C.

14. A methane conversion system comprising:
a source that produces a gas stream having a methane concentration from about 100 ppm to about 10,000 ppm;
oxic soil containing methanotrophic bacteria, wherein the methanotrophic bacteria are capable of oxidizing at least a portion of the methane to carbon dioxide; and
a gas distribution system configured to receive the gas stream and distribute the gas stream through the oxic soil.

15. The methane conversion system of claim 14, wherein the source is a lean-burn reciprocating engine.

16. The methane conversion system of claim 14, wherein the methanotrophic bacteria comprise Methylomonas aurantiaca, Methylomonas fodinarum, Methylomonas methanica, Methylobacter albus, Methylobacter marinus, Methylobacter whittenburyi, Methylococcus capsulatus, Methylococcus thermophiles, Methylomicrobium agile, Methylomicrobium album, Methylomicrobium pelagicum, or any combination thereof.

17. The methane conversion system of claim 14, further comprising a containment structure for the bed.

18. The methane conversion system of claim 14, wherein the bed has a bed depth of about 20 cm deep to about 50 cm deep.

19. The methane conversion system of claim 14, further comprising an underground vessel in which the gas stream is passed through and/or stored, wherein the underground vessel is downstream of the source and upstream of the gas distribution system.
